(19) United States
(12) Patent Application Publication
(10) Pub. No.: US 2026/0233601 A1
(43) Pub. Date: Aug. 13, 2026

(54) SYSTEM AND PROCESS FOR LIMITING OPERATION OF A VEHICLE BY AN UNAUTHORIZED OR IMPAIRED INDIVIDUAL

(71) Applicant: SUBSTANCE TECHNOLOGY HOLDING SWEDEN AB, Järfälla (SE)

(72) Inventor: Juan Miguel ARIAS, Solna (SE)

(21) Appl. No.: 19/535,875

(22) Filed: Feb. 10, 2026

(30) Foreign Application Priority Data
Feb. 10, 2025 (EP) 25156944

(51) Int. Cl. B60K 28/06; A61B 5/00; A61B 5/08; A61B 5/1171; A61B 5/16; A61B 5/18; B60R 16/023; G01N 33/497; G06F 8/65; G06V 20/59; G06V 40/16

(52) U.S. Cl. CPC B60K 28/06; A61B 5/002; A61B 5/082; A61B 5/1176; A61B 5/163; A61B 5/18; A61B 5/4845; B60R 16/0231; G01N 33/4972; G06F 8/65; G06V 20/597; G06V 40/172

(57) ABSTRACT

System and process for limiting operation of a vehicle by an unauthorized or impaired individual, the system comprising an alcometer configured to estimate the drug influence or content, in particular the blood alcohol content (BAC), of a user from a breath sample, and an interlock subsystem connected to the vehicle's control systems; wherein said alcometer wirelessly communicates with said interlock subsystem to limit operation of the vehicle if the estimated drug influence or content exceeds a predefined threshold; and the process comprising: estimating the drug influence or content, in particular the BAC, of a user from a breath sample with an alcometer; transmitting the estimate wirelessly from the alcometer to an interlock subsystem of the vehicle; and using the interlock subsystem to control the vehicle to limit its operation if the estimate exceeds a predefined threshold.

### CROSS REFERENCE TO RELATED APPLICATION

The present application claims priority to European Patent Application No. 25156944.8 filed on Feb. 10, 2025. The entire contents of the above-listed application are hereby incorporated by reference for all purposes

### TECHNICAL FIELD

The disclosure concerns a system for limiting operation of a vehicle by an unauthorized or impaired individual, the system comprising: an alcometer configured to estimate the drug influence or content, in particular blood alcohol content (BAC), of a user from a breath sample, and an interlock subsystem connected to the vehicle's control systems. The system is designed to prevent the operation of a vehicle by an unauthorized or impaired individual. This system is particularly useful for ensuring road safety by inhibiting individuals under the influence of drugs or alcohol from driving. The system comprises two main components: an alcometer and an interlock subsystem.

### BACKGROUND

The alcometer functions as a breathalyzer device, configured to estimate the drug influence or content, in particular the blood alcohol content (BAC), of a user through the analysis of a breath sample. This non-invasive method provides a reliable measurement of intoxication levels. The interlock subsystem, on the other hand, is connected to the vehicle's control systems, which may include ignition, fuel injection, or other critical operational components necessary for starting and running the vehicle.

### SUMMARY

The alcometer communicates wirelessly with the interlock subsystem, transmitting the BAC estimate. In one embodiment, the interlock subsystem may comprise a processing unit or firmware configured to independently evaluate the BAC estimate and determine whether it exceeds a predefined threshold, without relying on external systems (e.g., applications). If the measured or estimated drug influence or content, e.g. BAC, exceeds a predefined threshold (which may be determined by the interlock subsystem's internal logic in embodiments where the subsystem includes dedicated processing), the interlock subsystem engages, thereby restricting the vehicle's operation. This could manifest as preventing the ignition from engaging or ceasing fuel or power supply, effectively immobilizing the vehicle until a safe drug influence or content level is confirmed.

The technical effects of this system are profound. It significantly enhances road safety by preventing impaired individuals from operating vehicles, thus reducing the risk of accidents. Additionally, it ensures compliance with legal blood alcohol limits, offering a proactive measure against drunk driving. The use of wireless communication between components ensures reliability and ease of integration into various vehicle types equipped with electronic control systems, not limited to cars but potentially applicable to other vehicles as well, such as bikes, boats, ships, and airplanes.

The system may comprise a camera, wherein the interlock subsystem optionally comprises a facial recognition component, wherein the facial recognition component is connected to the camera and configured to authenticate the user of the alcometer captured by the camera. This variation of the system enhances the vehicle interlock system by incorporating a camera and facial recognition technology. This addition is designed to augment security and prevent unauthorized individuals from operating the vehicle. The camera captures images of the person using the alcometer, which are then analyzed by facial recognition software to authenticate the user's identity against a database of authorized users. If there is no match, the vehicle will not start, effectively preventing operation by unauthorized persons. The camera captures images or moving images (e.g., video) of the person using the alcometer. The captured video is stored in encrypted files on local storage (e.g., the alcometer's internal memory) or cloud-based storage (e.g., via the interlock subsystem's connection to remote services). Video processing is performed either locally on the alcometer or via edge computing on the interlock subsystem, enabling real-time facial recognition analysis. Transmitted video data is compressed and encrypted using protocols such as HTTPS/TLS to ensure secure transmission to remote services or devices.

This feature integrates seamlessly with the existing interlock subsystem, which controls critical functions such as ignition and fuel injection (or, in general, start and power supply). The system ensures that only recognized individuals can operate the vehicle, adding an extra layer of security beyond blood alcohol content (BAC) measurement. Optional features include verifying the correct positioning of the alcometer relative to the mouth, detecting foreign objects that may interfere with accurate BAC readings, and providing feedback to guide proper usage. The bonus effect of theft protection is notable, as facial recognition adds a significant barrier against vehicle theft by requiring biometric authentication for operation.

The interlock subsystem may optionally be implemented as a modular system comprising: a customized interlock component running on a computing device separate from the alcometer and the vehicle, in particular on a computing device of a user such as a smartphone, tablet, laptop, desktop computer, or wearable device (e.g., smartwatch) of a user, and an integrated interlock component integrated in the vehicle, wherein the integrated interlock component is connected to the vehicle's control systems and the alcometer wirelessly communicates with the customized interlock component, wherein the customized interlock component and the integrated interlock component are connected via an interlock connection, in particular an interlock interface provided by the integrated interlock component. The system may also be fully integrated into the vehicle's dashboard, where the customized interlock component (e.g., user interface, authentication tools) is displayed and operated via the vehicle's onboard display or infotainment system. This integration allows the alcometer to communicate directly with the vehicle's control systems through the dashboard, eliminating the need for external devices like smartphones.

This embodiment introduces an advanced modular design for the vehicle interlock system, enhancing its flexibility and adaptability. It incorporates two distinct yet interconnected components: a customized interlock component and an integrated interlock component. The customized interlock component is uniquely designed to operate on a separate computing device, such as a user's smartphone, while the integrated interlock component is seamlessly embedded within the vehicle itself. In an alternative configuration, the customized interlock component may run directly on the vehicle's dashboard interface, enabling the user to perform authentication (e.g., facial recognition) and view BAC estimates on the dashboard screen. This fully integrated design simplifies user interaction and reduces reliance on external devices.

This modular approach allows for efficient communication between the alcometer, which wirelessly transmits drug influence or content, e.g. blood alcohol content (BAC), data to the customized component on the smartphone. The smartphone then interacts with the vehicle's integrated interlock component via a dedicated interlock connection or interface. This setup ensures that the vehicle only operates when the drug influence or content level is within the permissible limit, thereby maintaining safety standards.

One of the significant advantages of this system is its scalability and ease of installation across various vehicle types. The use of a smartphone app for the customized component means that updates can be easily implemented without altering the vehicle's hardware, making the system more user-friendly and adaptable to future technological advancements. Additionally, this design promotes cost-effectiveness by leveraging existing smartphone technology, reducing the need for specialized hardware.

According to a first embodiment, the integrated interlock component may be a device electrically connected to a control line or control bus system of the vehicle and configured to wirelessly communicate with the customized interlock component. This embodiment further refines the modular interlock system introduced above by detailing the specific implementation of the integrated interlock component and its connection to the vehicle's control systems. This enhancement introduces a practical and efficient method for integrating the interlock subsystem into modern vehicles, leveraging existing automotive communication standards.

In this configuration, the integrated interlock component is realized as a compact device, such as a dongle, which is electrically connected to a critical vehicle control line or bus system. Optionally, the device may include a dedicated processing unit or embedded logic (e.g., firmware) to independently evaluate BAC estimates and enforce vehicle restrictions without relying on external applications. Examples of these connections include the ignition line, Controller Area Network (CAN) bus, or the On-Board Diagnostics (OBD-II) port, which provides direct access to the Engine Control Unit (ECU). This connection allows the integrated interlock component to monitor and control key vehicle functions, ensuring that operation is only permitted when safety conditions are met. The system is designed such that the alcometer does not directly interface with the dongle or integrated component. Instead, it wirelessly communicates with the customized interlock component running on a separate computing device, such as a smartphone. The customized interlock component then establishes communication with the integrated interlock component via an interlock connection or interface, enabling seamless data exchange between the alcometer, computing device (e.g. smartphone, tablet, or laptop), and vehicle systems. Optionally, wireless connectivity to the vehicle's control systems may be provided, such as through wireless OBD-II access. This eliminates the need for physical wiring in certain implementations, simplifying installation and reducing potential points of failure.

The advantages of this design are significant. By leveraging widely adopted automotive communication standards like CAN bus or OBD-II, the system ensures compatibility with a broad range of vehicles. Additionally, the use of a dongle or similar compact device makes installation straightforward, even for users without advanced technical expertise. The modular nature of the system also allows for cost-effective updates and maintenance, as software modifications can be implemented remotely via the smartphone app.

According to a second embodiment, the integrated interlock component may be a software component running on the same infrastructure as the vehicle's control system. This embodiment builds upon the modular interlock system introduced previously by introducing a significant advancement in the design of the integrated interlock component. In this configuration, the integrated interlock component is not a separate physical device but rather a software component that runs on the same hardware infrastructure as the vehicle's control systems. This approach eliminates the need for additional dedicated hardware, such as a dongle or external module, and instead leverages the existing computational resources of the vehicle's control systems.

By integrating the interlock functionality directly into the vehicle's control system, this design achieves a more seamless and efficient implementation. The software-based interlock component operates on the same CPU or processing unit as the vehicle's Engine Control Unit (ECU) or other control systems, enabling tighter integration with the vehicle's operational parameters. This direct integration allows for real-time monitoring and control of critical vehicle functions, ensuring that the interlock system responds swiftly and accurately to safety-critical inputs, such as the results from the alcometer.

One of the key advantages of this software-based approach is its scalability and adaptability. Since the integrated interlock component is implemented in software, it can be easily updated or modified without requiring physical changes to the vehicle's hardware. This makes the system more flexible and future-proof, as new features or safety protocols can be added through software updates. Additionally, the elimination of dedicated hardware reduces costs and simplifies installation, making the system more accessible for a wider range of vehicles. Furthermore, this design enhances reliability by minimizing the number of physical connections and potential points of failure. The use of shared hardware resources also ensures that the interlock system operates in harmony with the vehicle's existing control systems, reducing the likelihood of conflicts or malfunctions.

In another variation of the present disclosure, the determination whether the estimated drug influence or content, for example the estimated BAC (in the following, for readability, the abbreviation BAC is used as well for the specific example of blood alcohol content as for the general concept of a drug influence or content), exceeds a predefined threshold may be made by the interlock subsystem, in particular by an integrated interlock component. This feature ensures that the decision-making process is centralized within the interlock subsystem, improving the overall efficiency and reliability of the system. The determination may be made independently by the interlock subsystem's internal processing unit or firmware, in particular in embodiments where the interlock subsystem includes dedicated hardware or software logic to perform this evaluation without relying on external systems (e.g., applications). The predefined threshold may be obtained based on the current location of the vehicle, which can vary depending on regional legal limits or other factors. For this purpose, the system may include or be connected to a positioning system, such as GPS or similar technologies, to determine the vehicle's location accurately. In particular, the determination of whether the estimated influence or content exceeds the threshold is carried out by an integrated interlock component within the interlock subsystem. This component is responsible for processing data from various sources, including the BAC estimation and the current location of the vehicle, if applicable. The integration of positioning systems allows the system to adapt dynamically to different legal or regulatory requirements based on the vehicle's geographic location. For example, if the vehicle crosses into a jurisdiction with stricter BAC limits, the predefined threshold can be automatically adjusted accordingly. This feature achieves enhanced compliance with local laws and regulations, as well as increased accuracy in determining whether the driver is allowed to operate the vehicle. By incorporating real-time location data, the system may ensure that the BAC threshold is relevant to the specific context of the vehicle's operation. Additionally, the integration of positioning systems adds another layer of intelligence to the interlock subsystem, making it more versatile and adaptable to different operational environments.

The interlock subsystem may be configured to authenticate the alcometer before accepting a drug influence or content (e.g. BAC) estimate as a basis for the determination whether the estimated drug influence or content exceeds a predefined threshold. This introduces an enhanced security feature for the interlock system by incorporating alcometer authentication, ensuring that only authorized devices can provide valid blood alcohol content (BAC) estimates. This authentication process can be achieved through two primary methods: using a hardware address (MAC) or cryptographic certificates. The integration of user authorization adds another layer of security, potentially utilizing biometric authentication such as fingerprint or facial recognition to prevent unauthorized vehicle operation, thereby enhancing theft protection. The optional dual-layer security ensures that the vehicle can only be operated by authorized individuals who have provided a valid BAC estimate from an authenticated alcometer. Operational scenarios benefit from this feature as it prevents unauthorized access and ensures compliance with safety standards. This approach not only prevents drunk driving but also elevates vehicle security, making it a comprehensive solution for enhancing safety and preventing theft.

The interlock subsystem may further employ encryption for secure data transmission between the alcometer, interlock components, and remote services. This ensures data integrity and prevents tampering, enhancing the system's security against cyber threats.

In this context, the alcometer may further be configured to emit an indication of a confirmed test, wherein said indication comprises a representation of a temporary validation code for authentication purposes. This enhancement builds upon the authentication process outlined above by incorporating an additional layer of security through a temporary validation code emitted by the alcometer after confirming a test. This feature significantly strengthens the system's defenses against potential cyber threats and tampering attempts. Once a breath alcohol test is confirmed, the alcometer generates a unique temporary validation code, which serves as an extra authentication factor. This code is time-sensitive, meaning it is only valid for a brief period, thereby reducing the risk of replay attacks or unauthorized access. The integration of this feature ensures that even if an attacker attempts to intercept or spoof a valid BAC estimate, they would also need the corresponding temporary validation code to succeed. This enhancement not only bolsters the overall security of the system but also aligns with broader vehicle security objectives, particularly in preventing theft and ensuring driver safety. The implementation requires precise synchronization between the alcometer and the vehicle's system to validate codes efficiently without causing delays that might inconvenience the user. By introducing this temporary validation code, the system becomes more robust against evolving cyber threats while maintaining a seamless and efficient user experience. This layered approach to security underscores the importance of safeguarding both the authenticity and integrity of BAC estimates in automotive applications.

In an alternative embodiment, the alcometer may further be configured to signal a confirmed test to the interlock subsystem together with a timestamp of the test completion. This variation introduces an enhancement to the alcometer and interlock subsystem, focusing on adding a timestamp to the BAC test results. This feature ensures that each test is recent and authentic by including either an explicit or implicit timestamp, which helps prevent the use of old or falsified data. The alcometer not only measures blood alcohol content but also may capture visual evidence, such as videos or images, during the test. These visuals are augmented with timestamps to provide additional verification that the test was conducted properly and at the correct time. The integration of timestamps serves multiple purposes: it maintains accurate records, ensures the integrity of each test, and deters tampering by making it difficult to use manipulated data. Optionally, if moving images (e.g., video) are captured during the test, these are timestamped, encrypted, and stored in association with the BAC estimate for audit or verification purposes. Video files may use e.g. H.265 compression to minimize storage and bandwidth usage while maintaining sufficient resolution for facial recognition.

Optionally, the interlock subsystem may estimate, based on the estimated drug influence or content (e.g. BAC) and further stored user profile information, a future point in time when the user will return below the predefined threshold and inform the user accordingly. This introduces a sophisticated enhancement to an interlock subsystem by incorporating a predictive feature. This innovation allows the system not only to estimate a user's current Blood Alcohol Content (BAC) but also to predict when their BAC will drop below a predefined threshold, using stored user profile information such as weight and metabolism rate. The prediction process may involve an algorithm that considers various factors influencing alcohol metabolism, including body weight and individual drinking habits. This feature aims to provide users with clear guidance on when they can safely operate a vehicle again, thus enhancing safety by reducing the guesswork involved in estimating sobriety times.

At least parts of the interlock subsystem may be updated using over-the-air (OTA) update capability for remotely updating the interlock subsystem. This would allow for seamless remote updates to the interlock subsystem, enabling manufacturers or developers to improve, patch, or expand its capabilities without requiring physical access to the system. This OTA update feature ensures that the interlock subsystem remains up-to-date with the latest advancements in technology, security patches, and performance enhancements. By eliminating the need for physical access, this capability simplifies maintenance processes, reducing the time and effort required to keep the system current. Users benefit from enhanced convenience, as updates can be applied remotely without interrupting their use of the vehicle or requiring a visit to a service center. The integration of OTA updates also improves safety by enabling rapid deployment of critical security patches or software fixes that might address vulnerabilities or improve the accuracy and reliability of BAC measurements. This ensures that the system remains robust against potential tampering or exploitation, which is especially important given the critical role it plays in preventing impaired driving.

The interlock subsystem may optionally comprise an eye tracking component configured to monitor the attention and potential impairment of a driver of the vehicle, wherein the eye tracking component is configured to trigger a new alcometer test by the user. The integration of an eye-tracking component into the interlock subsystem can represent a significant advancement in vehicle safety by monitoring driver attention and potential impairment beyond traditional blood alcohol content (BAC) measurement. This technology aims to enhance safety by identifying signs of fatigue or distraction through eye movements, which can be indicative of impaired driving. The eye-tracking system can work in tandem with the existing alcometer, potentially triggering a new breathalyzer test if it detects anomalies in the driver's attention patterns. This dual-layered approach ensures that drivers are not only sober but also alert and focused on the road.

In an extended embodiment, the interlock subsystem may be connected to a remote monitoring service and configured to transmit estimated drug influence or content (e.g. BAC) values and optionally data recorded from a camera or an eye tracking component to the remote monitoring service. The connection to the remote monitoring service allows for the transmission of estimated Blood Alcohol Content (BAC) values, providing insights into whether a driver is under the influence of alcohol. The remote service can also optionally receive data from additional components such as a camera or an eye-tracking system, which monitor the driver's behavior and attention levels. This setup enables comprehensive oversight by recording not only BAC levels but also capturing visual data on the driver's state through the camera and tracking eye movements for signs of fatigue or distraction. Furthermore, details about user interactions with the alcometer, including the date, time, location, and unique identifiers like hardware addresses or digital signatures, may be transmitted to the remote service. These details help in verifying the authenticity and integrity of the data. The integration of real-time monitoring capabilities allows authorities or other stakeholders to intervene promptly if impaired driving is detected, enhancing road safety. The estimated values and optionally data recorded from a camera (including video files compressed with H.265 and encrypted via TLS) or an eye tracking component may be transmitted to the remote monitoring service.

In a further extended embodiment, the interlock subsystem may be connected to a remote authorization service and configured to receive access permissions associated with the alcometer, with particular drug influence or content (e.g. BAC) levels, with an authenticated user, with the vehicle, or with the location of the vehicle. This embodiment outlines an advanced interlock subsystem integrated with a remote authorization service, designed to dynamically manage access permissions for vehicle operation based on multiple factors: The system may connect to both remote authorization and monitoring services, enabling real-time communication. This integration allows for dynamic adjustments of access permissions based on data such as BAC levels, user authentication, vehicle identity, and location. Permissions can be stored locally on the interlock subsystem or requested from the remote service with each user interaction. This flexibility ensures that the system can operate both online and offline while maintaining up-to-date security measures. The system supports authenticated users, potentially using biometrics or passwords to ensure only authorized individuals can operate the vehicle. Multiple user profiles may exist for shared vehicles, each with specific permissions. There may be access permissions associated with the alcometer, with particular drug content levels, with an authenticated user (including facial recognition via video), with the vehicle, or with the location of the vehicle. Utilizing positioning technology, the system may adjust permissions based on geographical location, possibly adhering to regional alcohol laws. It may handle potential outages by defaulting to safe access denial until connectivity resumes. The system may employ encryption and secure communication methods to protect sensitive data transmitted to remote services, ensuring privacy and preventing unauthorized access. A seamless interface, possibly through an app or vehicle control panel, may allow drivers to view current permissions and restrictions without interrupting their experience. The system is designed to handle large-scale operations, such as managing company fleets, with robust infrastructure to support high data traffic and ensure quick response times. Comprehensive logging and auditing capabilities can track permission changes and accesses, essential for security and regulatory compliance

The disclosure also concerns a process for limiting operation of a vehicle by an unauthorized or impaired individual, the process comprising: estimating the drug influence or content, e.g. the blood alcohol content (BAC), of a user from a breath sample with an alcometer; transmitting the (e.g. BAC) estimate wirelessly from the alcometer to an interlock subsystem of the vehicle, in particular via a computing device such as a smartphone, tablet, laptop, desktop computer, or wearable device (e.g., smartwatch); and using the interlock subsystem to control the vehicle to limit its operation if the estimate exceeds a predefined threshold. The process is designed to prevent unauthorized or impaired individuals from operating a vehicle. Central to this process is the estimation of drug influence or content, in particular blood alcohol content (BAC), through an alcometer, which analyzes a breath sample provided by the user. This step ensures that the system can accurately assess whether the individual is under the influence of alcohol. Once the estimate is obtained, it is wirelessly transmitted from the alcometer to an interlock subsystem integrated into the vehicle. Wireless transmission enhances the system's convenience and reliability, eliminating the need for cumbersome wired connections and ensuring seamless communication between components.

The interlock subsystem then utilizes the received estimate to determine whether to limit the vehicle's operation. If the estimated drug influence or content (e.g. BAC) exceeds a predefined threshold—typically set in accordance with legal or regulatory limits—the interlock subsystem takes action to restrict the vehicle's functionality. This may include immobilizing the engine, restricting acceleration, or activating alerts to deter unsafe operation. By integrating these steps into a cohesive process, the disclosure ensures that vehicles are operated safely and responsibly, preventing accidents caused by impaired driving while also providing a robust framework for compliance with alcohol-related laws and regulations.

The technical effects of this process include enhanced road safety, reduced risk of alcohol-related accidents, and a reliable method for enforcing sobriety requirements. Additionally, the use of wireless communication ensures ease of installation and maintenance, making the system practical for widespread adoption across various types of vehicles. The predefined threshold can be customized to align with specific legal standards or organizational policies, offering flexibility in implementation while maintaining the core objective of preventing impaired driving.

The process may comprise authenticating the user providing the breath sample to the alcometer using facial recognition. In this refined process, before the estimation is conducted, the system first authenticates the user providing the breath sample through facial recognition technology. This authentication ensures that only authorized individuals can proceed with the drug influence or content test, thereby preventing unauthorized persons from attempting to operate the vehicle. The integration of facial recognition not only strengthens the security measures but also streamlines the process by ensuring that each step is conducted by the appropriate individual.

The technical effects and advantages of incorporating facial recognition into this process are manifold. Enhanced security is achieved by ensuring that vehicle operation is restricted to authorized users, thereby preventing potential misuse. This additional layer of authentication acts as a deterrent against unauthorized access and ensures compliance with safety protocols. Furthermore, by integrating facial recognition seamlessly into the existing estimation process, the system maintains user convenience while heightening overall security. This dual-layered approach—combining biometric authentication with drug influence or content assessment—significantly reduces the risk of impaired driving and enhances road safety.

The alcometer may emit an indication of a confirmed test when a sufficient breath sample has been received, wherein said indication comprises a representation of a temporary validation code for authenticating the particular test. The integration of an immediate indication upon receiving a sufficient breath sample and the generation of a temporary validation code significantly enhances the overall process outlined above. This innovation provides users with clear feedback, ensuring they know when their breath sample has been successfully captured. Such immediate confirmation improves user experience by eliminating uncertainty and reducing the need for repeated attempts. The use of a temporary validation code adds a robust layer of security to the system. This code serves as a verification mechanism, ensuring that the test results are genuine and not tampered with. The transient nature of the code—being valid only for a limited time—further enhances security by minimizing the potential for misuse if intercepted. This feature seamlessly integrates into the existing process, maintaining operational efficiency while bolstering reliability and security. The alcometer's indication can manifest through various outputs, such as visual cues, audible signals, or digital displays, ensuring accessibility and clarity for all users. The temporary validation code may for example be numerical, alphanumerical, or a QR code, offering flexibility in verification methods. By enhancing both user feedback and data security, this embodiment strengthens the system's ability to confidently and accurately control vehicle access based on BAC readings, thereby upholding its primary function of preventing impaired driving with added assurance of test integrity.

The determination whether the drug influence or content (e.g. BAC) estimate exceeds the predefined threshold can optionally be performed by the interlock subsystem, in particular by an integrated interlock component. This ensures that the critical decision-making step is handled by a dedicated part of the system, thereby improving integration with the vehicle's overall systems. The use of an integrated component improves reliability and consistency, as it minimizes potential errors and enhances seamless interaction with other vehicle functions. Additionally, this setup may allow for faster decision-making processes. For example, the determination may be performed independently by the interlock subsystem's internal processing unit or firmware, in particular in embodiments where the interlock subsystem includes dedicated hardware or software logic to perform this evaluation without relying on external systems (e.g., applications). The benefits of this integrated approach include enhanced safety through reduced error likelihood, improved system performance due to dedicated components, and more efficient operation. This refinement underscores the importance of a reliable and cohesive system in preventing impaired driving while maintaining ease of use and robust functionality.

The process may comprise using eye tracking on a driver of the vehicle between alcometer tests and triggering a new alcometer test by the user depending on the recognized attention and potential impairment of the driver. Building on the foundational process outlined above, which involves estimating drug influence or content, in particular blood alcohol content (BAC), from a breath sample, transmitting this data wirelessly to an interlock subsystem, and controlling vehicle operation based on a predefined threshold, the present embodiment introduces an additional layer of safety and monitoring. This enhancement incorporates eye tracking technology to continuously assess the driver's attention and potential impairment levels between alcometer tests. This system includes continuous monitoring of the driver's eye movements. This technology is designed to detect signs of drowsiness, distraction, or other indicators of potential impairment that could be linked to alcohol consumption. If the eye tracking system identifies anomalies in the driver's behavior, such as erratic eye movements or decreased attention span, it can automatically initiate a new alcometer test. This ensures that any possible increase in BAC levels is promptly detected and addressed. The eye tracking data may feed into the interlock subsystem, providing additional insights to enhance the accuracy of impairment assessments. This integration maintains the system's overall efficiency while improving its reliability.

### BRIEF DESCRIPTION OF THE FIGURES

Referring now to the drawings, wherein the figures are for purposes of illustrating the present disclosure and not for purposes of limiting the same:

FIG. 1 schematically shows a comprehensive system 1 designed to prevent impaired driving. The vehicle 2 is at the heart of this setup, housing a control system 3 that ensure safe operation. The user/driver 4 operates the vehicle 2 and is monitored by the alcometer 5, a breathalyzer device that measures blood alcohol content (BAC). This alcometer 5 communicates using a wireless connection 6 via Bluetooth technology to the smartphone 7, which acts as an intermediary in the system 1.

The smartphone 7, in turn, sends this BAC data to a dongle 8 plugged into the vehicle's OBD-II port via a second wireless connection 9. The dongle 8 facilitates bidirectional communication between the smartphone 7 and the vehicle's control system 3. The control system 3 then implements safety measures based on the received BAC levels, such as immobilizing the vehicle 2 if the BAC exceeds legal limits.

FIG. 2 schematically shows an enhanced version of the system 10 with a more streamlined architecture. The vehicle 11 remains central, housing the control systems 12 necessary for implementing safety protocols. The user/driver 13 is monitored by the alcometer 14, which now enjoys a direct wireless connection 15 to the vehicle's control system 12. This direct link eliminates the need for intermediary devices like a smartphone or dongle, enhancing communication efficiency.

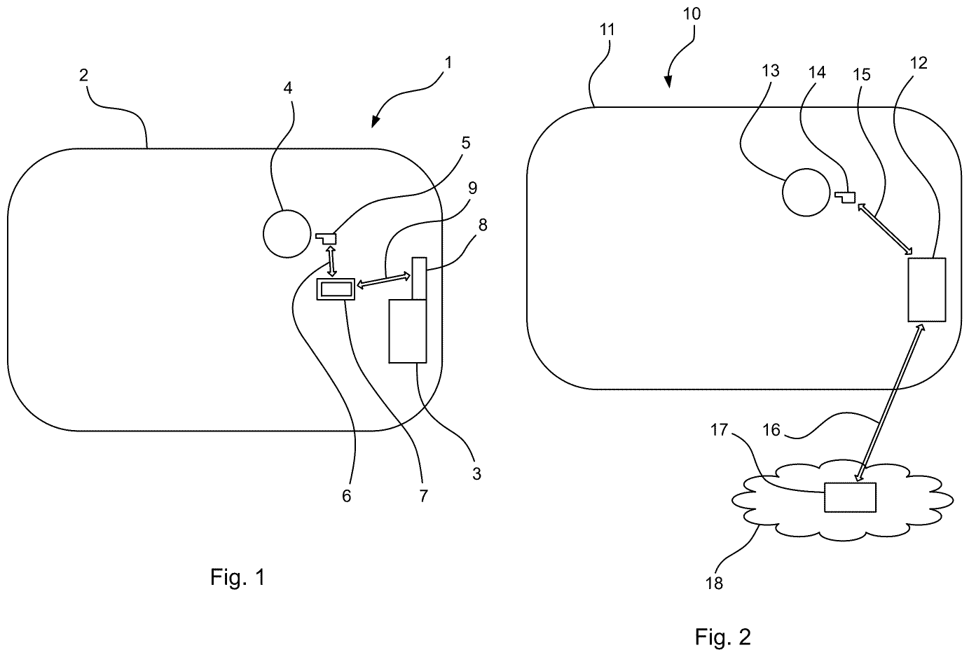

### DETAILED DESCRIPTION

The vehicle's control system 12 then communicates via a wireless uplink 16 with a remote monitoring and authorization service 17 hosted in the cloud 18. This service provides real-time oversight of the vehicle's status and the driver's BAC, authorizing or restricting vehicle operation based on predefined criteria. This setup ensures not only immediate implementation of safety protocols but also remote monitoring for enhanced security.

The system employs encryption protocols for all wireless communications between the alcometer, interlock subsystem, and remote services to prevent tampering and ensure data integrity.

The system implements user-friendly interfaces, such as smartphone applications and in-car displays, to enhance usability and streamline interaction with the interlock subsystem, ensuring that users can easily understand and operate the system without technical expertise. In embodiments where the system is fully integrated into the vehicle's dashboard, the interlock subsystem's user interface (e.g., facial recognition prompts, BAC readings, and authentication requests) is displayed directly on the dashboard screen. This eliminates the need for external devices and ensures a seamless user experience.

Users are provided with comprehensive training and educational resources to ensure proper understanding of system functionality and best practices for safe operation, including instructions on proper alcometer usage, interpretation of system alerts, and response to vehicle restrictions.

1. System for limiting operation of a vehicle by an unauthorized or impaired individual, the system comprising:
an alcometer configured to estimate the drug influence or content of a user from a breath sample, and
an interlock subsystem connected to the vehicle's control systems;
wherein said alcometer wirelessly communicates with said interlock subsystem to limit operation of the vehicle if the estimated drug influence or content exceeds a predefined threshold.

2. System according to claim 1, wherein the system comprises a camera, wherein the interlock subsystem comprises a facial recognition component, wherein the facial recognition component is connected to the camera and configured to authenticate the user of the alcometer captured by the camera.

3. System according to claim 1, wherein the interlock subsystem is implemented as a modular system comprising:
a customized interlock component running on a computing device separate from the alcometer and the vehicle, and
an integrated interlock component integrated in the vehicle,
wherein the integrated interlock component is connected to the vehicle's control systems and the alcometer wirelessly communicates with the customized interlock component
wherein the customized interlock component and the integrated interlock component are connected via an interlock connection.

4. System according to claim 3, wherein the integrated interlock component is a device electrically connected to a control line or control bus system of the vehicle and configured to wirelessly communicate with the customized interlock component.

5. System according to claim 3, wherein the integrated interlock component is a software component running on the same infrastructure as the vehicle's control system.

6. System according to claim 1, wherein the determination whether the estimated drug influence or content exceeds a predefined threshold is made by the interlock subsystem.

7. System according to claim 6, wherein the interlock subsystem is configured to authenticate the alcometer before accepting a drug influence or content estimate as a basis for the determination whether the estimated drug influence or content exceeds a predefined threshold.

8. System according to claim 7, wherein the alcometer is further configured to emit an indication of a confirmed test, wherein said indication comprises a representation of a temporary validation code for authentication purposes.

9. System according to claim 7, wherein the alcometer is further configured to signal a confirmed test to the interlock subsystem together with a timestamp of the test completion.

10. System according to claim 6, wherein the interlock subsystem estimates, based on the estimated drug influence or content and further stored user profile information, a future point in time when the user will return below the predefined threshold and inform the user accordingly.

11. System according to claim 1, wherein at least parts of the interlock subsystem can be updated using over-the-air (OTA) update capability for remotely updating the interlock subsystem.

12. System according to claim 1, wherein the interlock subsystem comprises an eye tracking component configured to monitor the attention and potential impairment of a driver of the vehicle, wherein the eye tracking component is configured to trigger a new alcometer test by the user.

13. System according to claim 1, wherein the interlock subsystem is connected to a remote monitoring service and configured to transmit estimated drug influence or content values and optionally data recorded from a camera or an eye tracking component to the remote monitoring service.

14. System according to claim 1, wherein the interlock subsystem is connected to a remote authorization service and configured to receive access permissions associated with the alcometer, with particular drug influence or content levels, with an authenticated user, with the vehicle, or with the location of the vehicle.

15. A process for limiting operation of a vehicle by an unauthorized or impaired individual, the process comprising:
estimating the drug influence or content of a user from a breath sample with an alcometer;
transmitting the estimate wirelessly from the alcometer to an interlock subsystem of the vehicle; and
using the interlock subsystem to control the vehicle to limit its operation if the estimate exceeds a predefined threshold.

16. The process according to claim 15, wherein the process comprises:
authenticating the user providing the breath sample to the alcometer using facial recognition.

17. The process according to claim 15, wherein the alcometer emits an indication of a confirmed test when a sufficient breath sample has been received, wherein said indication comprises a representation of a temporary validation code for authenticating the particular test.

18. The process according to claim 15, wherein the determination whether the estimate exceeds the predefined threshold is performed by the interlock subsystem.

19. The process according to claim 15, wherein the process comprises using eye tracking on a driver of the vehicle between alcometer tests and triggering a new alcometer test by the user depending on the recognized attention and potential impairment of the driver.
